(19) United States
(12) Patent Application Publication
(10) Pub. No.: US 2007/0105937 A1
(43) Pub. Date: May 10, 2007

(54) Indole-3-propionamide and derivatives thereof

(76) Inventors: Miguel Pappolla, Flowood, MS (US); Burkhard Poeggeler, Gottigen (DE)

Correspondence Address: JONES, WALKER, WAECHTER, POITEVENT, CARRERE;& DENEGRE, L.L.P., 5TH FLOOR, FOUR UNITED PLAZA, 8555 UNITED PLAZA BOULEVARD, BATON ROUGE, LA, 70809, US

(21) Appl. No.: 11/018,900

(22) Filed: Dec. 21, 2004

Related U.S. Application Data
(60) Provisional application No. 60/532,108, filed on Dec. 22, 2003.

(51) Int. Cl. A61K 31/405

(52) U.S. Cl. 514/419

(57) ABSTRACT

Indolepropionamide (IPAM) and related compounds, pharmaceutical or dietary compositions thereof and methods of using said compounds are disclosed for use as a preventative or therapeutic treatment for many conditions related to oxidative damage. Oxidative damage increases in aging and age related disorders and is widespread in many neurodegenerative conditions including Alzheimer's disease, Parkinson's disease and others. Indolepropionamide is a potent anti-oxidant and anti-aging molecule, with superior properties as compares to previously known compounds.

This application claims priority to U.S. Provisional Application No. 60/532,108, filed on Dec. 22, 2003.

Throughout this application, various publications are referenced, many in parenthesis. Full citations for these publications are provided at the end of each part of the application. The disclosures of these publications in their entireties are hereby incorporated by reference in this application.

### STATEMENT REGARDING FEDERALLY SPONSORED RESEARCH OR DEVELOPMENT

The subject matter of this application was made with support from the United States Government, National Institutes of Health Grant No. AG16783. The government may have certain rights in this application.

### FIELD OF THE INVENTION

The present invention relates to the synthesis of indole-3-propionamide (IPAM) and related compounds, including, but not limited to, pharmaceutical or nutritional compositions thereof, and methods of using the compounds and compositions for the following:

1) Preventative and therapeutic indications, including, but not limited to, inflammatory, degenerative, genetic, free-radical mediated, neoplastic (malignant or pre-malignant), age-related or age-associated, traumatic or infectious diseases affecting an organ or system or the whole organism, human or animal including, but not limited to: systemic illnesses including central nervous system disorders (e.g., atherosclerosis, degenerative joint disease, Alzheimer's disease, Parkinson's disease and other neurodegenerative conditions including, but not limited to, amyotrophic lateral sclerosis, Huntington's disease, Lewy body disease, epilepsy) and trauma to any bodily area including the head; promotion of neuronal or cellular regeneration in acute, subacute or chronic injuries or conditions afflicting humans or animals including, but not limited to, the nervous system; inflammatory and free-radical mediated disorders including, but not limited to, aging and age-associated conditions such as those mentioned above, and including other disorders such as atherosclerosis or osteoporosis; vascular or ischemic diseases of the nervous system that include, but are not limited to, stroke, vascular (ischemic) dementia and migraine; proliferative disorders and related conditions affecting humans or animals including cellular atypia, dysplasia, pre-malignant and malignant stages of acute, subacute or chronic conditions; and prevention of degeneration of nerve cells (neurodegeneration) in the acute, subacute or chronic phase of cerebral injuries including, but not limited to, trauma or acute, subacute or chronic disorders of the nervous system such as neurodegenerative or ischemic disorders and epilepsy. The method comprises administering an effective amount of IPAM or salts thereof as an active ingredient and may include an suitable carrier thereof to a human or animal subject in need thereof to prevent or treat the above mentioned disorders that specifically include, but are not limited to, the mentioned conditions. In all of the conditions described above free radicals are known to play a role at some point in the pathogenesis by either causing or amplifying damage to biomolecules.

2) A method for delaying, inhibiting or treating normal or pathological aging and age related conditions, in which it is known that free radicals are known to play a role at some point in the pathogenesis by either causing or amplifying damage to biomolecules, the method comprising administering an effective amount of IPAM to a live multi or unicellular organism including human, animal or plant.

3) A method comprising administering or adding an effective amount of IPAM or salts thereof as an active ingredient with or without a suitable carrier to nutritional products or derivatives of nutritional products for the prevention, inhibition or treatment of the disorders or injuries listed above afflicting unicellular or multicellular life or life-like form including, but not limited to, bacteria, fungi, or plants.

4) A method comprising administering or adding of an effective amount of IPAM or salts thereof as an active ingredient with or without a suitable carrier to nutritional products or derivatives of nutritional products, including but not limited to foodstuffs and nutritional supplements, or agricultural products for enhancing the health promoting properties of such products, for the preservation of the products themselves, or for the treatment or prevention of conditions or injuries affecting the products or the intended consumer of the products.

### BACKGROUND OF THE INVENTION

Oxidative damage increases in aging and age related disorders and is widespread in many neurodegenerative conditions including Alzheimer's disease, Parkinson's disease and others. In some of these conditions, clinical and epidemiological evidence suggest that anti-oxidants such as vitamin E may lower the risk or delay clinical milestones. Previous studies conducted by Pappolla et al. and Poeggeler et al. (Pappolla et al. 1998; Poeggeler et al 2001) have shown that anti-oxidants like melatonin may be useful in Alzheimer's disease and in other conditions characterized by oxidative (free-radical) damage. Experiments in a transgenic mouse model of Alzheimer's amyloidosis showed that melatonin inhibited the expected time dependent elevation of Aβ amyloid, ameliorated memory deficits and decreased the mortality in transgenic mice (data presented in the World Alzheimer's congress, 2000, Matsubara,et al. 2003).

Melatonin is a hormone which is synthesized and secreted primarily by the pineal gland and it acts both as a neurotransmitter and neurohormone. The hormone has the following structure: 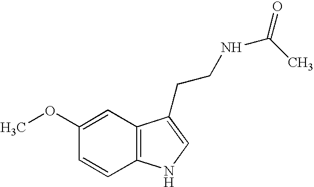
Being lipid soluble, melatonin rapidly crosses the blood brain barrier and other tissues. Once released from the pineal gland, which is highly vascularized, melatonin enters the general circulation and the cerebrospinal fluid (CSF). Melatonin acts on the central and peripheral nervous system as well as on peripheral endocrine target tissues and has been implicated in many human disorders. Some disorders are known to be linked to oxidative damage (free-radical damage) and others to chronobiologic abnormalities.

Interestingly, melatonin exhibits potent antioxidant and cellular protective (including neuroprotective) properties (Reiter, 1995), but, in contrast to conventional antioxidants, this hormone has a proposed physiologic role in the aging process (Pierpaoli, 1991; Pierpaoli et al., 1991) and decreased secretion of melatonin with aging is documented (Iguchi et al., 1982; Dori et al., 1994). In Alzheimer's disease, for example, there are reports of more profound reductions of melatonin secretion in populations with dementia than in non-demented controls (Souetre et al., 1989; Mishima et al., 1994). It has been suggested that altered secretion levels of the hormone may partially reflect the loss of daily variation in the concentration of melatonin in the pineals of elderly individuals and AD patients (Skene et al., 1990). Melatonin has also been shown to protect many cell types and tissues exposed to a large number of injuries including free-radical mediated, inflammation and others. Melatonin was shown to be effective in preventing death of cultured neuroblastoma cells as well as oxidative damage and intracellular Ca²⁺ increases induced by a cytotoxic fragment of amyloid beta-protein (Pappolla et al., 1997). The use of melatonin for its cytoprotective effect in enhancing survivability of cells that have been subjected to the cytotoxic effects of amyloid beta protein (an abnormal neurotoxic substance that deposits in brains with Alzheimer's disease and also causes oxidative stress) as well as for treating patients afflicted with Alzheimer's Disease is disclosed in U.S. Pat. No. 5,958,964 (Pappolla) and U.S. Pat. No. 6,274,615 (Pappolla et al.).

In addition, it has been reported that melatonin has oncostatic properties in a variety of cancers, the most studied being melatonin's effects on estrogen receptor positive breast cancers (Blask et al 1986; Gonzalez et al. 1991; Lisoni et al. 1993; Shellard et al.1989; Philo et al. 1988; see U.S. Pat. No. 5,196,435 of Clemens et al. and U.S. Pat. No. 5,272,141 of Fraschini et al.).

Research into the structure of indoles by a number of laboratories suggests that the antioxidant and neuroprotective features of these substances might be preserved in a spectrum of many related compounds. Some of these are structurally related, but due to modifications in their chemical structure, they have preserved antioxidant and neuroprotective features with more therapeutically advantageous properties. An example of one such indole structure is indole-3-propionic acid (IPA), which has the following structure: 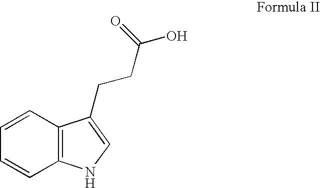
IPA exhibited more powerful antioxidant properties than melatonin (about a 6 fold increase) but in contrast to most antioxidants (including melatonin), it did not form pro-oxidant intermediates (Chyan et al., 1999). Significantly, IPA also has a longer half-life than melatonin (4.5 hr versus 20 minutes). Despite the mentioned advantages, there remains the fact that IPA is not as lipophilic as melatonin and blood brain barrier penetration for IPA is rather poor (BBB penetration ratio melatonin: IPA=100:1), requiring large doses of the compound to attain the desired neuroprotective effects. The use of IPA to prevent cytotoxic effects of amyloid beta protein, to treat fibrillogenic diseases, to decrease oxidation in biological samples, and to treat diseases or other conditions where free radicals and/or oxidative stress play a role was disclosed in U.S. Pat. No. 6,395,768 (Pappolla et el.) and is hereby incorporated by reference.

### SUMMARY OF THE INVENTION

In order to overcome the disadvantages of IPA, the inventors herein have discovered that IPAM, indole-3-propionamide (3-(3-indolyl)propionamide), its derivatives, analogs and related compounds have unique properties that are more effective for the disclosed uses than IPA and its derivatives. IPAM exhibits a three-dimensional structure of an amide resembling a “reversed melatonin” and integrates the best structural features of melatonin and IPA overcoming the main limitations of both drugs, i.e. poor BBB penetration and short half-live. The general structure of IPAM and its derivatives are represented by Formula III: 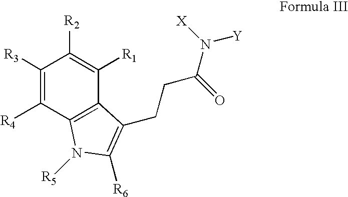
wherein X and Y are either hydrogen or an alkyl, aryl or arylalkyl group; and R₁ through R₆ are either hydrogen, a halogen, or a hydroxy, alkoxy, alkyl, aryl, alkoxyalkyl, or alkyaryl group.

The general structure of IPAM related compounds are represented by Formula IV: 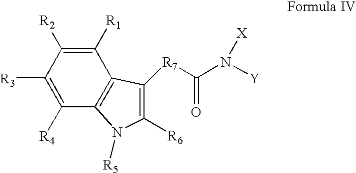
wherein X and Y are either hydrogen or an alkyl, aryl or arylalkyl group; R₁ through R₆ are either hydrogen, a halogen, or a hydroxy, alkoxy, alkyl, aryl, alkoxyalkyl, or alkyaryl group; and R₇ is either an alkyl, aryl or arylalkyl group.

When the term “IPAM” is used herein it is understood that IPAM analogs and/or derivatives and related compounds, and pharmaceutically suitable salts thereof, are included by the term.

In contrast to indole-3-propionic acid which bears a polar carboxyl group which is ionized at physiological pH carrying a negative charge, IPAM and its related compounds are non polar with sufficient lipophilicity (and amphiphilicity) to penetrate the BBB. In contrast to melatonin, IPAM and its related compounds are “reversed amides” lacking the methoxy group as an aromatic substituent. Melatonin is quickly metabolized in the liver by hydroxylation in para position to its large side chain (extensive first pass effect with rapid clearence) and excreted as the glucuronide or sulfate conjugate of 6-hydroxy-melatonin. IPAM and its related compounds, however, have a long half-live and can accumulate in mammalian tissues with its high stability and bioavailability due to the amide structure as illustrated.

The inventors herein initially discovered IPAM in rodent bile and have also observed endogenous IPAM in the gastrointestinal tract, brain and cerebrospinal fluid of mice and rats. Most significantly is IPAM lipophilic characteristics that allow this newly identified molecule to penetrate through many cell membrane compartments. Although indole-3-propionamide was already known as a molecular structure related to melatonin (Suzen et al 2001) its properties as documented herein were not previously recognized.

In the experiments detailed below, IPAM exhibits powerful free-radical scavenging properties like melatonin, IPA and other indole compounds but with superior pharmacokinetic properties making the compounds useful in a number applications including, but not limited to, increasing life span of human, animals and uni-cellular or multi-cellular organisms, but particularly in higher vertebrates and humans, in which both, penetration through biologic barriers and half life (IPAM has a better half life profile than other thus far known indole related structures) are paramount for therapy efficacy. These properties would make the compound useful for prevention and treatment of a large number of conditions, for use in health promoting strategies, and as a preservative or additive agent for foodstuffs, nutritional supplements or agricultural products.

### BRIEF DESCRIPTION OF THE DRAWINGS

FIG. 1 presents the results of a Thioflavin T spectrofluorometric analysis illustrating the inhibition of Aβ aggregation.

### PREFERRED EMBODIMENTS OF THE INVENTION

In the following detailed description of the preferred embodiments, reference is made to the accompanying drawings, which form a part hereof, and in which are shown by way of illustration specific embodiments in which the invention may be practiced. It is to be understood that other embodiments may be utilized and structural changes may be made without departing from the scope of the present invention.

Dosage and Formulation

IPAM and its related compounds can be administered by any means that produces contact of the active agent with the agent's site of action. Sites of action include the body of a subject or animal, a cell or living organism. Sites of action may also include, but are not limited to, foodstuffs, nutritional supplements or agricultural products. For any application, IPAM and its related compounds can be prepared by any conventional means available for use either alone or in conjunction with other pharmaceuticals, vitamins or cytoprotective or bioprotective agents. It can be administered totally alone, but the preferred means of administration is in conjuntion with a pharmaceutical carrier selected on the basis of the chosen route of administration and standard pharmaceutical practice. The pharmaceutical compositions of the invention may be adapted for oral, parenteral, rectal, transdermal and nasal administration, and may be in unit dosage form, as well known to those skilled in the pharmaceutical art. The active ingredient for oral administration in solid dosage forms includes, for example, tablets, capsules or powders, or in liquid dosage forms, such as aqueous or oily suspensions, disperse powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents, and preserving agents in order to provide a pharmaceutically elegant and palatable preparation. Tablets contain the active ingredient in admixture with non-toxic pharmaceutically acceptable excipients, which are suitable for manufacture of tablets, for example, inert diluents, such as calcium carbonate, sodium carbonate, lactose, calcium phosphate, or sodium phosphate; granulating disintegrating agents, e.g., maize starch, or alginic acid; binding agents, such as starch, gelatin, or acacia; and lubricating agents, for example, magnesium stearate, stearic acids or talc. The tablets may be uncoated or they may be coated by known techniques to delay disintegration and adsorption in the gastrointestinal tract. Thereby a sustained action over a longer period can be provided.

Formulations for oral use can also be presented as hard gelatin capsules wherein the active ingredient is mixed with an inert solid diluent, e.g., calcium carbonate, calcium phosphate, or kaolin, or as soft gelatin capsules wherein the active ingredient is mixed with an oil medium, such as arachis oil, liquid paraffin or olive oil.

Aqueous suspensions contain the active compound in admixture with excipients suitable for the manufacture of aqueous suspensions. Such excipients are suspending agents including but not limited to sodium carboxymethylcellulose, methylcellulose, hydroxypropylmethylcellulose, sodium alginate, polyvinylpyrrolidone, gum tragacanth, and gum acadia; dispersing or wetting agents, such as a naturally-occurring phosphatide, e.g., lecithin, or condensation products of an alkylene oxide with fatty acids, for example of polyoxyethylene stearate, or a condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monooleate, or a condensation product of ethylene oxide with partial esters derived from fatty acids and hexitol anhydrides, e.g., polyoxyethylene sorbitan monooleate. The aqueous suspensions can also contain one or more preservatives, for example ethyl, n-propyl, or p-hydroxy benzoate, one or more coloring agents, one or more flavoring agents, and one or more sweetening agents, such as sucrose, saccharin, or sodium or calcium cyclamate.

Disperse powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents, can also be present.

Syrups and elixirs can be formulated with sweetening agents, such as glycerol, sorbitol or sucrose. Such formulations may also contain a demulcent, a preservative and flavoring and coloring agents. The pharmaceutical compositions can be in the form of a sterile injectable preparation, for example, as a sterile injectable aqueous suspension. This suspension can be formulated according to the known art using those suitable dispersing or wetting agents and suspending agents, which have been mentioned above. The sterile injectable preparation can also be a sterile injectable solution or suspension in a non-toxic parenterally-acceptable diluent or solvent, for example, as a solution in 1,3-butane diol.

In general, a pharmacologically effective daily dose can be from about 0.01 mg to about 10 g per dose, bearing in mind, or course, that in selecting the appropriate dosage in any specific case, consideration must be given to the subject's weight, general health, metabolism, age and other factors which influence response to a drug.

The preferred embodiment of this invention is the provision of pharmaceutical compositions in dosage unit form, which comprise from about 0.5 mg to about 500 mg of a compound of the above formulae.

The optimization of prophylactic or therapeutic efficacy in administering IPAM or related compounds according to the method of the present invention, which optimization includes dosage, formulation for delivery (i.e., sustained release), administration schedule (i.e., intervals), can be determined by those of skill in the art with routine experimentation using conventional practices.

For non-in vivo administration (such as addition of IPAM or its related compounds as a food preservative), the optimization of the amount of the compound needed can be determined by those of skill in the art with routine experimentation using conventional practices.

It will also be appreciated that the actual preferred amount of IPAM or related compounds to be administered according to the present invention will vary according to the particular active forms of the compound (active forms include the use of IPAM structure as a pharmacophore, which is the active structure of the drug), the particular composition formulated, and the mode of administration. Many factors that may modify the action of IPAM or its related compounds can be taken into account by those skilled in the art e.g., body weight, sex, diet, time of excretion, condition of the subject, drug combinations, and reaction sensitivities and severities. Administration can be carried out continuously or periodically within the maximum tolerated dose. Optimal administration rates for a given set of conditions can be ascertained by those skilled in the art using conventional dosage administration tests.

Suitable routes of administration include systemic administration (because IPAM and its related compounds will cross the blood-brain barrier). Systemic administration includes parenteral and oral administration, for example, as discussed in further detail below.

The pharmaceutical compositions of the present invention also include compositions for delivery across cutaneous or mucosal epithelia including transdermal, intranasal, sublingual, buccal, and rectal administration. Such compositions may be part of a transdermal device, patch, topical formulation, gel, etc. with appropriate excipients. Thus, the compounds of the present invention can be compounded with a penetration-enhancing agent such as 1-n-dodecylazacyclopentan-2-one or the other penetration-enhancing agents disclosed in U.S. Pat. Nos. 3,991,203 and 4,122,170, which are hereby incorporated by reference in their entirety to describe penetration-enhancing agents which can be included in the transdermal or intranasal compositions of this invention.

The pharmaceutical compositions can be formulated so that for every 100 parts by weight of the composition there are present between 1 and 99 parts by weight of the active ingredient.

Having now generally described the invention, the same will be more readily understood through reference to the following examples, which is provided by way of illustration and is not intended to be limiting of the present invention.

### SYNTHESIS OF 3-(3-INDOLYL)PROPIONAMIDE

IPAM and its related compounds can be prepared by methods generally known to those skilled in the art or by novel methods described herein. The method described below is only one of many available to those skilled in the art and is not exclusive of other methods to arrive to the compounds. One such method for preparation of the active ingredient of the novel pharmaceutical compounds of the present invention will be illustrated by the following non-limitative specific examples:

A mixture of 30 g of indole-3-propionic acid and 10 ml of methanesulfonic acid in 200 ml of ethanol was stirred for 24 hours, poured into water, and extracted with ethylacetate. The ethylacetate solution was washed with NaHCO₃ solution and water and dried over magnesium sulfate. A solution of 800 mg of the crude product (indole-3-propionic acid ethyl ester) and 2 ml of hydrazine in 20 ml of ethanol was refluxed for 18 hours, and extracted with ethylacetate. The organic phase was washed with brine, dried over magnesium sulfate, and evaporated at reduced pressure to give with 93% yield the intermediate propanoic acid hydrazide as a solid. This material and 0.3 g of Raney nickel catalyst (W-4) in 25 ml of ethanol were refluxed for 2.5 hours. The solution was decanted and evaporated at reduced pressure and the residue chromatographed on silicia gel, eluting with ethylacetate, to give with 96% yield indole-3-propionamide.

Pharmaceutically suitable salts of the compound of the invention can be prepared by reacting the free acid precursor or base forms of the compound with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference in its entirety.

As described above, the compounds of the present invention are generally useful in the treatment of indications including, but not limited to:

Preventative and therapeutic indications, including, but not limited to, inflammatory, degenerative, genetic, free-radical mediated, neoplastic (malignant or pre-malignant), age-related or age-associated, traumatic or infectious diseases affecting an organ or system or the whole organism, human or animal including, but not limited to: systemic illnesses including central nervous system disorders (e.g., atherosclerosis, degenerative joint disease, Alzheimer's disease, Parkinson's disease and other neurodegenerative conditions including, but not limited to, amyotrophic lateral sclerosis, Huntington's disease, Lewy body disease, epilepsy) and trauma to any bodily area including the head; promotion of neuronal or cellular regeneration in acute, subacute or chronic injuries or conditions afflicting humans or animals including, but not limited to, the nervous system; inflammatory and free-radical mediated disorders including, but not limited to, aging and age-associated conditions such as those mentioned above, and including other disorders such as atherosclerosis or osteoporosis; vascular or ischemic diseases of the nervous system that include, but are not limited to, stroke, vascular (ischemic) dementia and migraine; proliferative disorders and related conditions affecting humans or animals including cellular atypia, dysplasia, pre-malignant and malignant stages of acute, subacute or chronic conditions; and prevention of degeneration of nerve cells (neurodegeneration) in the acute, subacute or chronic phase of cerebral injuries including, but not limited to, trauma or acute, subacute or chronic disorders of the nervous system such as neurodegenerative or ischemic disorders and epilepsy. The method of treatment comprising administering an effective amount of IPAM or salts thereof as an active ingredient and may include an suitable carrier thereof to a human or animal subject in need thereof to prevent or treat the above mentioned disorders that specifically include, but are not limited to, the mentioned conditions; Inhibiting or treating normal or pathological aging and age related conditions comprising administering an effective amount of IPAM to a live multi or unicellular organism including human, animal or plant; Administering or adding an effective amount of IPAM or salts thereof as an active ingredient with or without a suitable carrier to nutritional products or derivatives of nutritional products for the prevention, inhibition or treatment of the disorders or injuries listed above afflicting unicellular or multicellular life or life-like form including, but not limited to, bacteria, fungi, or plants; and administering or adding of an effective amount of IPAM or salts thereof as an active ingredient with or without a suitable carrier to nutritional products or derivatives of nutritional products, including but not limited to foodstuffs and nutritional supplements, or agricultural products for enhancing the health promoting properties of such products, for the preservation of the products themselves, or for the treatment or prevention of conditions or injuries affecting the products or the intended consumer of the products.

### EXAMPLE 1

An important feature of IPAM, as compared to other neuroprotective indoles such as melatonin or indolepropionic acid is the combination of both, longer half life and lipophilic properties as demonstrated in the following example:

Time dependent changes in indole levels in brain tissue (pg/mg protein) after administration of 0.5 mg/kg i.p. of drug in phosphate buffered saline to one month old male Sprague-Dawley rats (N=4 animals, mean+SD). Endogenous levels of melatonin in saline treated control animals were 25+10 pg/mg protein, endogenous levels of indolepropionate were 80+20 pg/mg protein. Significant amounts of endogenous IPAM in rat brain could not be detected. Brain tissue was homogenized 1:10 in phosphate buffer and extracted 1:1 with ethylacetate. Recovery for melatonin, indolepropionate and IPAM were 57+6, 69+12 and 80+4%. Indoles were measured by HPLC with electrochemical detection (20% methanol eluent, 990 mV oxidation potential, IPAM elutes at 18 minutes, melatonin at 21 minutes, indolepropionate at 36 minutes).

| Treatment | 30 minutes | 60 minutes | 120 minutes |
|---|---|---|---|
| Melatonin | 450 + 30 | 200 + 15 | 73 + 6 |
| Indolepropionate | 160 + 11 | 210 + 18 | 230 + 25 |
| IPAM | 480 + 46 | 520 + 50 | 570 + 69 |

### EXAMPLE 2

### Inhibition of Hydroxyl Radical Mediated Oxidative Damage to DNA By IPAM in Rat Forebrain Homogenate

Rat forebrain homogenate was incubated for sixty minutes with 3 mM hydrogen peroxide, 4 mM ferrous sulfate and 2 mM ADP to generate hydroxyl radicals. The concentrations of each indole compound to reduce oxidative DNA damage by 50%, the IC 50 values, are given as means+standard deviations for N=6 different determinations. The IC 50 values were calculated by running six experiments each at 10 increasing concentrations ranging from 0.01 to 100 micromolar of melatonin, indole-3-propionic acid and indole-3-propionamide. DNA damage was examined by measuring the formation of 8-hydroxydeoxyguanosine with HPLC and electrochemical detection in the presence and absence of the hydroxyl radical scavengers.

| Treatment | IC 50 |
|---|---|
| Melatonin | 1.40 + 0.16 μM |
| Indole-3-propionic acid | 7.46 + 0.80 μM |
| IPAM | 0.18 + 0.03 μM |

### EXAMPLE 3

### Inhibition of the Aging Process in a Rotifer Model (Philodina) By Melatonin, Indolepropionate and IPAM

Free-radicals are involved in the aging process and since IPAM is a powerful cytoprotective agent, we tested IPAM for its property to inhibit the aging process. The following experiments illustrate that not only was IPAM devoid of toxicity, but it dramatically extended the life span of rotifers, an established model of aging. The potency of IPAM exceeded that of melatonin and IPA and was far superior to any other compound thus far described in the literature. (*=p<0.05, statistically significant difference to control).

| | Median life | | | Maximum life | |
|---|---|---|---|---|---|
| | span in days | Increase in % | | span in days | Increase in % |
| Treatment | (50% survival) | of control | | (90% survival) | of control |
| Control (0.1% DMSO) | 23.4 +− 0.6 | | | 27 | |
| 0.5 μM melatonin | 28.0 +− 1.5 | +19% | (N.S.) | 35 | +30% |
| 0.5 μM | 30.0 +− 1.7 | +28% | (*) | 39 | +37% |
| indolpropionic acid | | | | | |
| 0.5 μM IPAM | 31.8 +− 1.7 | +31% | (*) | 42 | +55% |
| Control (0.1% DMSO) | 25.9 +− 0.8 | | | 30 | |
| 5 μM melatonin | 33.5 +− 1.5 | +29% | (*) | 42 | +40% |
| 5 μM | 33.8 +− 1.2 | +30% | (*) | 41 | +37% |
| indolpropionic acid | | | | | |
| 5 μM IPAM | 36.3 +− 1 | +40% | (*) | 45 | +50% |
| Control (0.1% DMSO) | 25.0 +− 0.9 | | | 30 | |
| 50 μM melatonin | 28.1 +− 0.7 | +12% | (N.S.) | 31 | +3% |
| 50 μM | 24.3 +− 1.0 | −3% | (N.S.) | 30 | +−0% |
| indolpropionic acid | | | | | |
| 50 μM IPAM | 28.8 +− 1.5 | +15% | (*) | 34 | +13% |

### EXAMPLE 4

### Synergistic Effects on Longevity of Rotifers (Philodina) By Melatonin, Indolepropionate and IPAM with Ascorbate and Trolox

Ascorbate and trolox were administered at a concentration of 100 micromolar, the indoles were given at a concentration of 5 micromolar. Presented is the mean and the SD of ten experiments on individual rotifers with * being significantly different at a p level of less than 0.05 and N.S. being not statistically significant.

| | Medium | Increase in % | | Maximum | Increase in % |
|---|---|---|---|---|---|
| Treatment | Life Span | of control | | Life Span | of control |
| Control (0.1% DMSO) | 24.5 +− 0.8 | | | 30 | |
| 100 μM Ascorbate | 24.3 +− 0.6 | −1% | (N.S.) | 28 | −7% |
| 100 μM Trolox | 26.4 +− 0.7 | +8% | (N.S.) | 30 | +−0% |
| Ascorbate and Trolox | 28.9 +− 1.1 | +18% | (*) | 36 | +20% |
| Ascorbate in 0.1% DMSO | 26.4 +− 0.7 | | | 30 | |
| Ascorbate and Melatonin | 37.7 +− 1.8 | +43% | (*) | 48 | +60% |
| Ascorbate and | 39.7 +− 1.5 | +50% | (*) | 48 | +60% |
| Indolpropionate | | | | | |
| Ascorbate and IPAM | 44.6 +− 1.7 | +68% | (*) | 54 | +80% |
| Trolox in 0.1% DMSO | 29.3 +− 1.1 | | | 34 | |
| Trolox and Melatonin | 38.4 +− 1.7 | +31% | (*) | 48 | +41% |
| Trolox and | 42.4 +− 1.5 | +45% | (*) | 51 | +50% |
| Indolpropionate | | | | | |
| Trolox and | 46.1 +− 1.6 | +57% | (*) | 57 | +67% |
| Indolpropionamide | | | | | |
| Ascorbate and Trolox | 28.8 +− 0.9 | | | 34 | |
| Ascorbate, Trolox and | 42.4 +− 2.0 | +47% | (*) | 54 | +59% |
| Melatonin | | | | | |
| Ascorbate, Trolox and | 44.0 +− 2.0 | +53% | (*) | 56 | +65% |
| Indolpropionate | | | | | |
| Ascorbate, Trolox and | 50.4 +− 1.7 | +75% | (*) | 60 | +77% |
| IPAM | | | | | |

### EXAMPLE 5

### Inhibition of Aβ Aggregation By IPAM and IPA

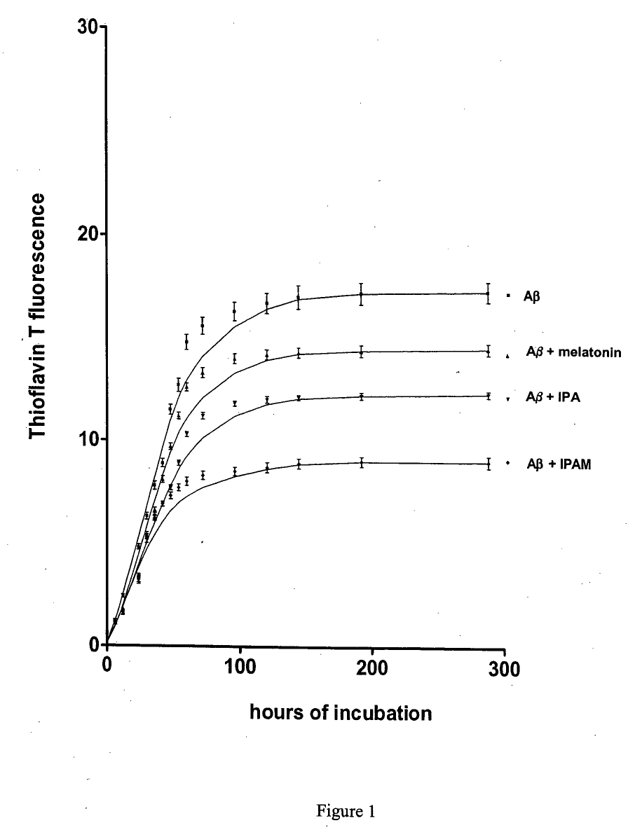

Like melatonin and IPA, IPAM was a potent inhibitor of Aβ aggregation. FIG. 1 illustrates the results of thioflavin T fluorescence experiments performed with Aβ1-40 showing the effects of IPAM as compared with melatonin and IPA.

The ThT was performed as previously described. Briefly, 5 μl were obtained from each sample after incubation and added to 2 ml of a glycine-NaOH buffer (50 mM), pH 9.2, containing 2 μM thioflavin T. Fluorescence intensities were measured at excitation 435 nm and emission 485 nm in a Hitachi F-2000 fluorescence spectrophotometer. A time scan of fluorescence intensity was performed and three measurements were obtained after the decay reached a plateau at 200, 220, and 240 seconds. These measurements were averaged after subtracting the background fluorescence of 2.0 μM thioflavin T in the blank buffers. The compounds used in the experiments did not exhibit significant fluorescence within the regions of interest at any time-point. All measurements were done in triplicate. Aqueous stock solutions of 1 mM of each compound were made by first preparing a 10 mM suspension in 1 N HCl and then by completely dissolving them in 100 mM phosphate buffered saline at pH 7.4 (1:10, v:v) and re-adjusting the pH to 7.4 with 1 N NaOH. Solutions of Aβ were prepared by dissolving 2.2 mg of the peptide in 1 ml of 50 mM bicarbonate buffer at pH 9.6. 50 μl aliquots of this solution were lyophilized and stored at −80° C. until needed. Working stock solutions of the peptide (500 μM concentration) were prepared in HPLC-grade water immediately prior to the experiments. In the experimental samples, Aβ was further diluted 1:1 with phosphate buffered saline (pH 7.4, 100 mM) to which each of the compounds or equivalent volumes of buffer solution were added. The final concentration of Aβ in each sample was 250 μM and the compound:Aβ molar ratios were 1:1.

### REFERENCES

Blask D. E. and Hill S. M. (1986) Effects of melatonin on cancer: studies on MCF-7 human breast cancer cells in culture. J. Neural Transm. Suppl. 21,433-449
Chyan Y.-J., Poeggeler B., Omar R. A., Chain D. G., Frangione B., Ghiso J. and Pappolla M. A. (1999) Potent neuroprotective properties against the Alzheimer beta-amyloid by an endogenous melatonin-related structure, indole-3-propionic acid. J. Biol. Chem. 274, 21937-21942
Dori D., Casale G., Solerte S. B., Fioravanti M., Migliorati G., Cuzzoni G. and Ferrari E. (1994) Chrono-neuroendocrinological aspects of physiological aging and senile dementia. Chronobiologia 21, 121-126
Gonzalez R., Sanchez A., Ferguson J. A., Balmer C., Daniel C., Cohn A. and Robinson W. A. (1991) Melatonin therapy of advanced human malignant melanoma. Melanoma Res. 1, 237-243
Iguchi H., Kato K. and Ibayashi H. (1982) Melatonin serum levels and metabolic clearance rate in patients with liver cirrhosis. J. Clin. Endocrinol. Metab. 54, 1025-1027
Lissoni P., Barni S., Rovelli F., Brivio F., Ardizzoia A., Tancini G., Conti A. and Maestroni G. J. (1993) Neuroimmunotherapy of advanced solid neoplasms with single evening subcutaneous injection of low-dose interleukin-2 and melatonin: preliminary results. Eur. J. Cancer 29,185-189
Matsubara E., Bryant-Thomas T., Pacheco Quinto J., Henry T. L., Poeggeler B., Herbert D., Cruz-Sanchez F., Chyan Y. J., Smith M. A., Perry G., Shoji M., Abe K., Leone A., Chain D G, Neria E., Grundke-Ikbal I. Wilson G. L., Ghiso J., Williams C., Refolo L. M. and Pappolla M. A. (2003) Melatonin increases survival and inhibits oxidative and amyloid pathology in a transgenic model of Alzheimer's disease. J. Neurochem. 85, 1101-1108.
Mishima K., Okawa M., Hishikawa Y., Hozumi S., Hori H. and Takahashi (1994) Morning bright light therapy for sleep and behavior disorders in elderly patients with dementia. Acta Psychiat. Scand. 89, 1-7
Pappolla M. A., Sos M., Omar R. A., Bick R. J., Hickson-Bick D. L. M., Reiter R. J., Efthimiopoulos S and Robakis N. K. (1997) Melatonin prevents death of neuroblastoma cells exposed to the Alzheimer's amyloid pertide. J. Neurosi. 17, 1683-1690
Pappolla M. A., Bozner P., Soto C., Shao H., Robakis N. K., Zagorski M., Frangione B. and Ghiso J. (1998) Inhibition of Alzheimer β-fibrillogenesis by melatonin. J. Biol. Chem. 273, 7185-7189.
Philo R. and Berkowitz A. S. (1988) Inhibition of Dunning tumor growth by melatonin. J. Urol. 139, 1099-1102,
Pierpaoli W. (1991) The pineal gland: a circadian or seasonal aging clock? Aging (Milano) 3, 99-101
Pierpaoli W., Dall'Ara A., Pedrinis E. and Regelson W. (1991) The pineal control of aging. The effects of melatonin and pineal grafting on the survival of older mice. Ann. N Y Acad. Sci. 621, 291-313.
Poeggeler B., Miravalle L., Zagorski M. G., Wisniewski T., Chyan Y.-J., Zhang Y., Shao H., Bryant-Thomas T., Vidal R., Frangione B., Ghiso J. and Pappolla M. A. (2001) Melatonin reverses the profibrillogenic activity of apoplipoprotein E4 on the Alzheimer amyloid Aβ peptide. Biochemistry 40, 14995-15001.
Reiter J. (1995) The role of the neurohormone melatonin as a buffer against macromolecular oxidative damage. Neurochem Int. 27, 453-460.
Shellard S. A., Whelan R. D. and Hill B. T. (1989) Growth inhibitory and cytotoxic effects of melatonin and its metabolites on human tumour cell lines in vitro. Br. J. Cancer 60, 288-290.
Skene D. J., Vivien-Roels B., Sparks D. L., Hunsaker J. C., Pevet P., Ravid D. and Swaab D. F. (1990) Daily variation in the concentration of melatonin and 5-methoxytryptophol in the human pineal gland: effect of age and Alzheimer's disease. Brain. Res. 528, 170-174
Souetre E., Salvati E., Krebs B., Belugou J. L. and Darocurt G. (1989) Abnormal melatonin response to 5-methoxypsoralen in dementia. Am. J. Psychiatry 146, 1037-1040
Suzen S., Ates-Alagoz Z., Demircigil B. T. and Ozkan S. A. (2001) Synthesis and analytical evaluation by voltammetric studies of some new indole-3-propionamide derivatives. Farmaco 56, 835-840.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed:

1) A pharmaceutical composition comprising an effective amount of:
(a) a compound of the general formula, 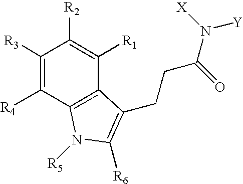
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, and R₁ through R₆ are independently selected from the group consisting of hydrogen, a halogen, a hydroxyl group, an alkoxy group, an alkyl group, an aryl group, an alkoxyalkyl group, and an alkyaryl group; and
(b) a pharmaceutically acceptable carrier.

2) A pharmaceutical composition comprising an effective amount of:
(a) a compound of the general formula, 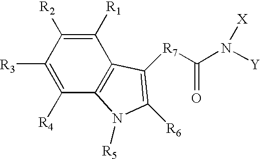
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, R₁ through R₆ are independently selected from the group consisting of hydrogen, a halogen, a hydroxyl group, an alkoxy group, an alkyl group, an aryl group, an alkoxyalkyl group, and an alkyaryl group, and R₇ is selected from the group consisting of an alkyl group, an aryl group and an arylalkyl group; and
(b) a pharmaceutically acceptable carrier.

3) A pharmaceutical composition comprising an effective amount of:
(a) indole-3-propionamide or a pharmaceutically acceptable salt thereof; and
(b) a pharmaceutically acceptable carrier.

4) A method of treating or delaying the onset of a condition where free radicals and/or oxidative stress contribute to the pathogenesis, comprising administering to a subject an effective amount of a compound of the general formula, 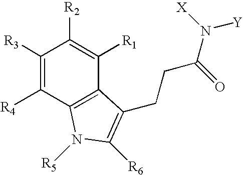
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, and R₁ through R₆ are independently selected from the group consisting of hydrogen, a halogen, a hydroxyl group, an alkoxy group, an alkyl group, an aryl group, an alkoxyalkyl group, and an alkyaryl group.

5) The method of claim 4, wherein said administration is carried out systemically.

6) The method of claim 4, wherein the condition is caused by a disease selected from the group consisting of inflammatory disease, degenerative disease, genetic disease, free radical mediated disease, malignant neoplastic disease, pre-malignant neoplastic disease, and age-associated disease.

7) The method of claim 6, wherein the disease was caused by trauma.

8) The method of claim 6, wherein the disease was caused by infection.

9) The method of claim 4, wherein the subject is human.

10) The method of claim 9, wherein the condition is a systemic illness.

11) The method of claim 10, wherein the systemic illness is a central nervous system disorder.

12) The method of claim 10, wherein the systemic illness is selected from the group consisting of atherosclerosis, degenerative joint disease, Alzheimer's disease, and Parkinson's disease.

13) The method of claim 10, wherein the systemic illness is a neurodegenerative condition.

14) The method of claim 13, wherein the neurodegenerative condition is selected from the group consisting of amyotrophic lateral sclerosis, Huntington's disease, Lewy body disease, and epilepsy.

15) The method of claim 9, wherein the condition was caused by trauma to a bodily area.

16) The method of claim 15, wherein the trauma is to the head.

17) The method of claim 4, wherein the condition requires the promotion of cellular regeneration.

18) The method of claim 17, wherein the condition requires the promotion of neuronal regeneration.

19) The method of claim 4, wherein the condition is a nervous system disorder.

20) The method of claim 19, wherein the nervous system disorder resulted from vascular disease.

21) The method of claim 20, wherein the nervous system disorder resulted from ischemia.

22) The method of claim 19, wherein the nervous system disorder is selected from the group consisting of stroke, dementia and migraine.

23) A method for delaying, inhibiting or treating normal or pathological aging and/or age related conditions comprising administering to a live organism an effective amount of a compound of the general formula, 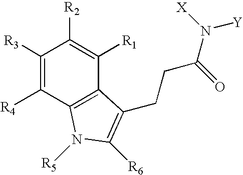
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, and R₁ through R₆ are independently selected from the group consisting of hydrogen, a halogen, a hydroxyl group, an alkoxy group, an alkyl group, an aryl group, an alkoxyalkyl group, and an alkyaryl group.

24) A method of claim 23, wherein the administration is accomplished by adding the effective amount of the compound to a nutritional product.

25) The method of claim 24, wherein the live organism is a plant.

26) The method of claim 24, wherein the live organism is an animal.

27) The method of claim 26, wherein the live organism is human.

28) A method for enhancing the health promoting properties of a nutritional product that is consumed by an organism comprising the adding to the nutritional product an effective amount of a compound of the general formula, 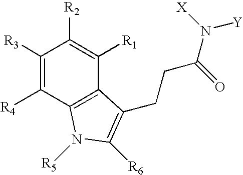
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, and R₁ through R₆ are independently selected from the group consisting of hydrogen, a halogen, a hydroxyl group, an alkoxy group, an alkyl group, an aryl group, an alkoxyalkyl group, and an alkyaryl group.

29) A method for preserving a nutritional product against the effects of oxidative and/or free radical damage comprising the adding to the nutritional product an effective amount of a compound of the general formula, 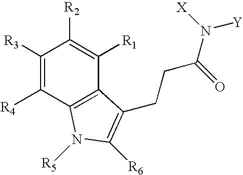
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, and R₁ through R₆ are independently selected from the group consisting of hydrogen, a halogen, a hydroxyl group, an alkoxy group, an alkyl group, an aryl group, an alkoxyalkyl group, and an alkyaryl group.

30) A method of treating or delaying the onset of a condition where free radicals and/or oxidative stress contribute to the pathogenesis, comprising administering to a subject an effective amount of a compound of the general formula, 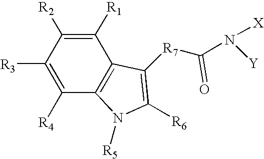
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, R₁ through R₆ are independently selected from the group consisting of hydrogen, a halogen, a hydroxyl group, an alkoxy group, an alkyl group, an aryl group, an alkoxyalkyl group, and an alkyaryl group, and R₇ is selected from the group consisting of an alkyl group, an aryl group and an arylalkyl group.

31) The method of claim 30, wherein said administration is carried out systemically.

32) The method of claim 30, wherein the condition is caused by a disease selected from the group consisting of inflammatory disease, degenerative disease, genetic disease, free radical mediated disease, malignant neoplastic disease, pre-malignant neoplastic disease, and age-associated disease.

33) The method of claim 32, wherein the disease was caused by trauma.

34) The method of claim 32, wherein the disease was caused by infection.

35) The method of claim 30, wherein the subject is human.

36) The method of claim 35, wherein the condition is a systemic illness.

37) The method of claim 36, wherein the systemic illness is a central nervous system disorder.

38) The method of claim 36, wherein the systemic illness is selected from the group consisting of atherosclerosis, degenerative joint disease, Alzheimer's disease, and Parkinson's disease.

39) The method of claim 36, wherein the systemic illness is a neurodegenerative condition.

40) The method of claim 39, wherein the neurodegenerative condition is selected from the group consisting of amyotrophic lateral sclerosis, Huntington's disease, Lewy body disease, and epilepsy.

41) The method of claim 35, wherein the condition was caused by trauma to a bodily area.

42) The method of claim 41, wherein the trauma is to the head.

43) The method of claim 30, wherein the condition requires the promotion of cellular regeneration.

44) The method of claim 43, wherein the condition requires the promotion of neuronal regeneration.

45) The method of claim 30, wherein the condition is a nervous system disorder.

46) The method of claim 45, wherein the nervous system disorder resulted from vascular disease.

47) The method of claim 46, wherein the nervous system disorder resulted from ischemia.

48) The method of claim 45, wherein the nervous system disorder is selected from the group consisting of stroke, dementia and migraine.

49) A method for delaying, inhibiting or treating normal or pathological aging and/or age related conditions comprising administering to a live organism an effective amount of a compound of the general formula, 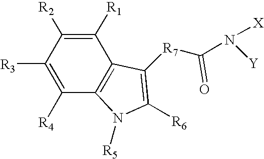
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, R₁ through R₆ are independently selected from the group consisting of hydrogen, a halogen, a hydroxyl group, an alkoxy group, an alkyl group, an aryl group, an alkoxyalkyl group, and an alkyaryl group, and R₇ is selected from the group consisting of an alkyl group, an aryl group and an arylalkyl group.

50) A method of claim 49, wherein the administration is accomplished by adding the effective amount of the compound to a nutritional product.

51) The method of claim 50, wherein the live organism is a plant.

52) The method of claim 50, wherein the live organism is an animal.

53) The method of claim 52, wherein the live organism is human.

54) A method for enhancing the health promoting properties of a nutritional product that is consumed by an organism comprising the adding to the nutritional product an effective amount of a compound of the general formula, 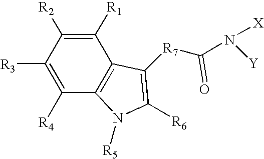
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, R₁ through R₆ are independently selected from the group consisting of hydrogen, a halogen, a hydroxyl group, an alkoxy group, an alkyl group, an aryl group, an alkoxyalkyl group, and an alkyaryl group, and R₇ is selected from the group consisting of an alkyl group, an aryl group and an arylalkyl group.

55) A method for preserving a nutritional product against the effects of oxidative and/or free radical damage comprising the adding to the nutritional product an effective amount of a compound of the general formula, 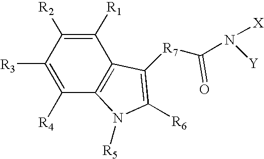
or a pharmaceutically acceptable salt thereof, wherein X is selected from the group consisting of hydrogen, an alkyl group, an aryl group and an arylalkyl group, Y is selected from the group consisting of hydrogen, an alkyl group, an aryl group and an arylalkyl group, R₁ through R₆ are independently selected from the group consisting of hydrogen, a halogen, a hydroxyl group, an alkoxy group, an alkyl group, an aryl group, an alkoxyalkyl group, and an alkyaryl group, and R₇ is selected from the group consisting of an alkyl group, an aryl group and an arylalkyl group.
